(19) United States
(12) Patent Application Publication
(10) Pub. No.: US 2022/0080142 A1
(43) Pub. Date: Mar. 17, 2022

(54) HEAT AND MOISTURE EXCHANGER DEVICE (HME) WITH FILTERING

(71) Applicant: Alan Henry SHIKANI, Ruxton, MD (US)

(72) Inventor: Alan Henry SHIKANI, Ruxton, MD (US)

(21) Appl. No.: 17/475,967

(22) Filed: Sep. 15, 2021

Related U.S. Application Data
(60) Provisional application No. 63/204,124, filed on Sep. 15, 2020.

(51) Int. Cl. A61M 16/04; A61M 16/10

(52) U.S. Cl. CPC A61M 16/047; A61M 16/0468; A61M 16/1045

(57) ABSTRACT

A heat and moisture exchanger device (HME) mounted on an end of a tracheotomy tube. The heat moisture exchange device has, in parallel with reticulated polyurethane foam filter, an N95 filter material that is customized in a wafer shape so that to precisely fits inside the housing of the HME, with a good seal and avoiding air leakage. Airflow is redirected inside the HME in a turbulent fashion, replicating Brownian Motion and the phenomena of Impaction, Interception, and Diffusion that are typically found in a HEPA filter, and enhancing filtration of air that is breathed by tracheotomized patients, hence protecting these patients from inhaling airborne germs and viruses, including COVID 19.

### RELATED APPLICATIONS

This application is related to Provisional Application 63/204,124 filed Sep. 15, 2020, the entirety of which is incorporated herein by reference.

### BACKGROUND

The current COVID-19 pandemic presents a unique challenge for tracheotomized patients. In the normal population, respiratory viruses, including COVID-1, access the body through the nose and mouth. Wearing face masks, particularly N95 masks, are an effective way to reduce infection to an individual and to minimize transmission to other surrounding people. Tracheostomized patients on the other hand, are obligatory neck breathers and are not adequately protected by traditional N95 surgical face masks as these masks do not have a good fit around the tracheostomy tube and do not provide an effective airflow seal around the tube (FIGS. 1a, 1b, 1c). Therefore, current traditional N95 surgical face masks do not provide adequate filtration or protection to the tracheostomized patients against COVID-19, influenza and/or other viruses. Furthermore, if tracheotomized patients happen to get infected with COVID-19, they present a potential danger to people around them through airborne transmission by coughing or exhaling the virus.

It is also important to note that tracheostomized patients are in general at higher risk for poor outcomes with COVID-19 due to comorbidities, such as chronic pulmonary disease and a tendency for alveolar atelectasis due to the loss of upper airway resistance, impaired mucociliary function and mucosal irritation from breathing colder, dryer and unfiltered air. To start with, because these patients are obligatory neck breathers, air that is inhaled does not pass through the nose and reaches the lungs with substantially lower temperature, humidification, and without particulate filtering. When dryer, cooler, and unfiltered air reaches the lungs, detrimental health effects occur, including thickened mucus, impaired mucociliary transport, and mucosal damage. Aggregates of dried mucus may fall, occlude deeper airways, and promote atelectasis or infection. HME use helps mitigate these effects. FIGS. 2a, 2b, 2c a bronchoscopy views of the trachea and upper bronchi of tracheotomized patients breathing unfiltered, dry, and cold air through the tracheostomy tube and without the protection of an HME with a filter. Visible in the views are thickened mucus and crusting, mucosal inflammation, and infection.

The problem is substantial as there are about 125,000 tracheotomies performed every year in the United States (U.S.) according to the Centers for Medicare & Medicaid Services (CMS) and there are an estimated 300,000 patients who live with permanent tracheostomies in the U.S. Since a tracheotomy is a procedure that is commonly performed worldwide, the numbers are proportional to the world population and patients who live with permanent tracheostomies worldwide is likely to be in the millions, so a solution to this problem is urgent.

### BACKGROUND ON THE COVID-19 VIRUS

The coronavirus disease of 2019 (“COVID-19”) is an infectious disease caused by severe acute respiratory syndrome coronavirus 2 (“SARS-CoV-2”). The disease was first identified in December 2019 in Wuhan, the capital of China's Hubei province, and has since spread globally, resulting in the ongoing 2019-2021 coronavirus pandemic. Common symptoms include fever, cough, and shortness of breath. Other possible symptoms include muscle pain, sputum production, diarrhea, sore throat, loss of smell, and abdominal pain. While the majority of cases result in mild symptoms, some progress to viral pneumonia and multi-organ failure, and eventually death.

As of September 2021, more than 39.8 million COVID-19 cases have been reported in the US with 646,000 deaths; and more than 220 million cases have been reported worldwide with 4.56 million deaths. Even though effective vaccines have been developed to fight COVID-19, only 49% of Americans have been vaccinated to date, and about 26.1% of the world population has received at least one dose of a COVID-19 vaccine. More ominously, only 1% of people in low-income countries have received at least one dose. To complicate things, the virus continues to mutate, and new variants (such as delta, mu) are causing more people to be infected even though they are fully vaccinated. We are still learning about how effective the different vaccines are against new variants of the COVID-19 virus, and how long the vaccine immunity lasts. What we know is that the delta and mu variants of the coronavirus appear to cause more severe illness than earlier variants and that the variants are extremely contagious; delta spreads as fast and as easily as chickenpox. Since tracheotomized patients cannot benefit from traditional masks, they are left utterly vulnerable and defenseless.

### BACKGROUND ON FILTRATION

The COVID-19 virus is approximately 0.125 micron (or 125 nanometers) in diameter; however, it often travels in biological aerosols which range in size from 0.5-3.0 micron. It is primarily spread during close contact and by small droplets produced when people cough, sneeze, or talk.

The National Institute for Occupational Safety and Health (NIOSH) has ratings for respirators of 95, 99, or 100 percent filtration efficiency. Major functional issues in the design and engineering of masks and respirators include fit and filtration. Most research to date has focused on filtration. NIOSH ratings for respirators of 95, 99, or 100 percent filtration efficiency are based on the percentage of 0.3 μm particles that do not penetrate the test filter. Fit is also extremely important but less is known about issues regarding inward face seal leakage and other aspects of respirator fit.

Powered Air-Purifying Respirators (PAPRs) are Personal Protective Equipment (PPE) devices are currently the ultimate protection devices against COVID-19; however, these devices are very expensive and hard to get. The devices consist of an air blowing motor and a plastic suit, which is fitted with High Efficiency Particulate Air (HEPA) filters that supply filtered air to a positive-pressure hood.

N95 respirators are also effective in protecting against COVID-19 airborne infections. N95 respirators are more readily available and more comfortable for the user, requiring less respiratory work and they are currently considered the standard protective device for health care workers.

We begin our background review on filtration by reviewing the design and mechanism of action of HEPA filters and by exploring the alternative types of filters which are more readily available. The U.S. Department of Energy use the term HEPA to refer to a filtering specification for suppliers of filtration products. The specification is based on how effective the filtration products are at particle removal. HEPA filters include a complicated mix of filaments and fibers carrying a static charge, which lures various microbes and particles similar to a magnet. Particles traveling through the air filtration system are captured and retained within the filter. Additionally, an effect known as Brownian Motion occurs causing particles in certain media states (such as fluid) to bounce around and become trapped.

HEPA filters remove from the air that passes through 99.97% of particles that have a diameter greater than or equal in size to 0.3 microns (ASME standard). In reality, HEPA filters are also efficient at capturing particles whose diameter is less than 0.3 microns which, in part because of Brownian motion, are actually even easier to capture and filter. HEPA filters function like a net: if a particle is smaller than the holes in the net, it gets through; so, the smaller the particle, the harder it is to capture in theory. This logic works for larger objects like marbles and reflects how HEPA filters work for particles greater than 0.3 microns in diameter. These particles either cannot fit through the filter, or their inertia causes them to hit the filter's fibers—processes called “Impaction” and “Interception”. During Impaction, larger particles are unable to avoid fibers when following the curving contours of the air stream and smash into and are forced to embed into one of these fibers directly; this effect increases with diminishing fiber separation and with higher airflow velocity. During Interception, particles following a line of flow in the air stream come within one radius of a fiber and adhere to it.

The common assumption that a HEPA filter acts like a sieve where particles smaller than the largest opening can pass through is incorrect; HEPA filters are also designed to target much smaller pollutants and particles. In addition to Impaction and Interception for the larger particles, these smaller particles are trapped by a process called Diffusion. During Diffusion, small particles collide with gas molecules, especially those below 0.1 μm in diameter, and these particles are thereby impeded and delayed in their path through the filter. Diffusion raises the probability that a particle will be stopped by either Interception or Impaction. This process is more dominant at lower airflow speeds.

For very small particles—e.g. less than 0.3 microns—these particles have such little mass that they actually get bounced around like a pinball when they hit gas molecules, and they move through the filter in random zigzag patterns—a phenomenon known as Brownian Motion.

These very small particles are small enough to fit through HEPA filters if they flew straight, but because they move in zigzag patterns, they end up hitting the filter fibers and getting stuck. This phenomenon of Brownian Motion applies to particles less than 0.3 microns in size, while “traditional” filtering through Impaction and Interception works on particles greater than 0.3 microns in size. Together, Impaction, Interception, and Diffusion allow HEPA filters to catch particles that are both larger and smaller than 0.3 microns in size. Diffusion predominates below the 0.1 micron diameter particle size, while Impaction and Interception predominate above the 0.4 micron diameter particle size. Particles closer to 0.3 microns in size are the hardest particles to capture—researchers call this the most penetrating particle size (MPPS). Near the MPPS, both Diffusion and Interception/Impaction are comparatively inefficient. Because this is the “weakest” point in the filter's performance, the HEPA specifications use the retention of particles near this size (0.3 microns) to classify the filter.

In summary, HEPA air filters are extremely more effective at capturing particles that are less than 0.3 microns in size, including in theory the COVID-19 virus, whose diameter is approximately 0.1 microns.

It is important however to point out that COVID-19 is a relatively new virus and little objective data is available about the true filtration effectiveness of current commercial devices (including HEPA) against this virus. What we know is that while the COVID-19 virus is approximately 0.125 microns in diameter, it often travels in biological aerosols which range in size from 0.5-3.0 micron. In current clinical practice, N95 surgical face masks are considered among the most effective types of face protection currently available generally against COVID-19. National Institute for Occupational Safety and Health (NIOSH) ratings for N95 respirators as having 95% filtration efficiency are based on the percentage of 0.3 μm particles that do not penetrate this type of filter.

To date, there are no filters available on the market to protect tracheotomized patients against COVID-19. Adding an N95 material inside the HME device in parallel to the reticulated polyurethane foam should provide the necessary additional filtration. This novel HME with N95 filtration hereby described is the first device designed to attach to the tracheostomy tube, combining the advantages of the N95 filtration and Brownian motion of reticulated polyurethane foam.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIGS. 1a, 1b, 1c are photos of a tracheotomized patient with a traditional face mask.

FIGS. 2a, 2b, 2c are bronchoscopy views of the trachea and upper bronchi of tracheotomized patients.

FIGS. 3a, 3b are a cross-section diagram and a photo of an HME with N95 wafer filter and polyurethane foam in accordance with an embodiment.

FIG. 4 is a view of turbulent airflow inside an HME according to an embodiment.

FIG. 5a is an image of foam before reticulation (left), after reticulation (right).

FIG. 5b is a drawing of a structure and properties of reticulated foams.

FIG. 6a is a graph comparison of airflow resistance inside the Shikani HME, the Shikani HME+N95 (90 Plus) according to an embodiment, and the Mallinckrodt HME.

FIG. 6b is a graph comparison of airflow resistance inside the Shikani HME, the Shikani HME+N95 (150 Plus) according to an embodiment, and the Mallinckrodt HME.

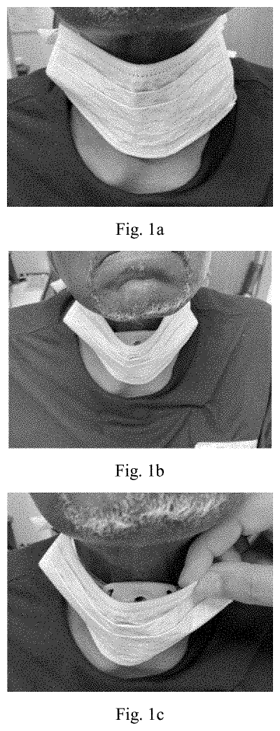

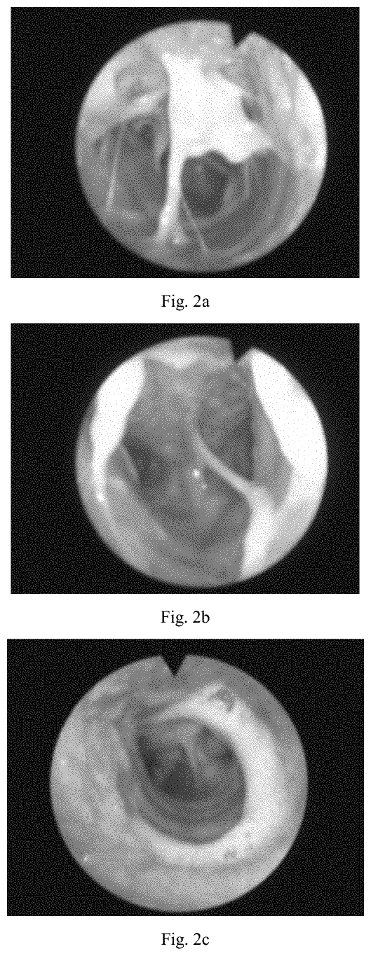

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

One or more embodiments are directed to a Heat and Moisture Exchanger (HME) device with a filter material customized in a wafer shape to precisely fit inside the housing of the HME in parallel with a reticulated polyurethane foam filter with a good seal and avoiding air leakage, to enhance filtration of air breathed by tracheotomized patients. In some embodiments, the filter material is N95 filter material. Thereby, the HME device with filter protects these patients from inhaling airborne germs and viruses, including COVID-19 and other germs and viruses. In some embodiments, the filter material is N99 or N100 filter material.

In addition, the HME device with filter prevents direct finger contact to the cannula of the tracheotomy tube (since the HME covers the tracheostomy tube) hence minimizing the risk of direct surface transmission of the virus to the tracheotomized patient.

In addition, by filtering viruses from exhaled air, this HME with filter would also protect people surrounding a tracheotomized patient who happens to be infected with COVID-19 and other germs and viruses.

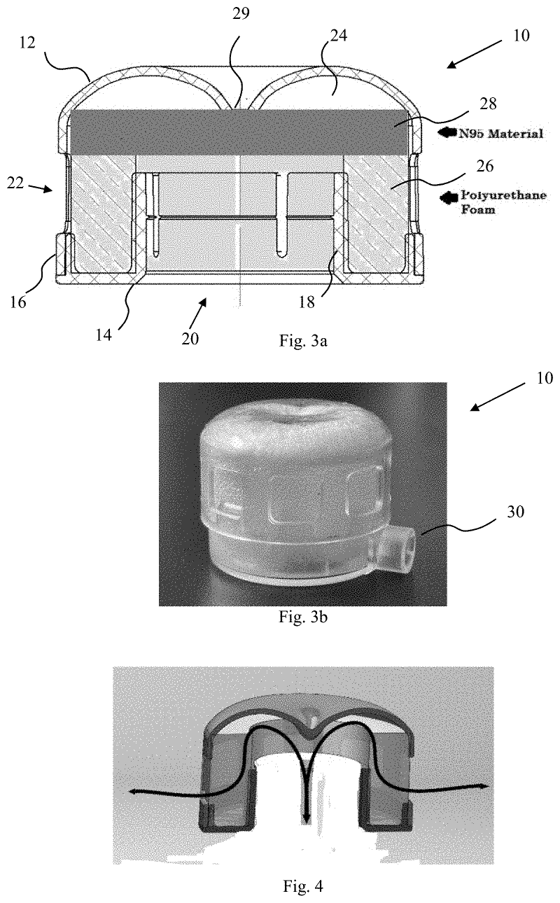

In FIG. 3a, a housing 10 is configured to be received on an end of a tracheotomy tube wherein exhaled and inhaled air may move into and out of the housing. The housing has a domed frontal wall 12. A bottom circular panel 14 is joined to the circumferential walls 16. A circular opening 20 formed centrally in the bottom panel 14. A plurality of legs 18 are connected to the bottom panel 14, the legs extending upwardly within the circular opening 20 toward the domed frontal wall 12. The individual legs 18 are separated from the adjoining leg 18 by a narrow slit wherein the legs have a degree of flexibility. The legs 18 are substantially parallel to the circumferential walls 16.

A plurality of spaced-apart openings 22 are formed in the circumferential walls 16. Dead space 24 is formed within the housing 10, preferably bounded by the domed frontal wall 12.

A filter media 26 is formed as a sheet which is disposed completely around the housing between the interior of the circumferential wall 16 and the legs 18 extending upwardly from the bottom panel 14. In this manner, the filter media 26 covers all of the spaced-apart openings 22 so that air moving into or out of the housing must traverse the filter media. It has been found that porous reticulated ester-type polyurethane foam having a pore size of 65 pores per inch is satisfactory as a filter media 26. Pore sizes are available from 40 ppi to 90 ppi with 50 ppi to 70 ppi being most common. The filter media may not be a foam but could be a filter paper. The filter media is impregnated with a hygroscopic material. Calcium chloride has been found to be a satisfactory hygroscopic material. In some embodiments, filter media 26 is shaped as a right cylinder with an opening through the center.

N95 or higher filter material 28 is formed as a sheet which is on top of the filter media 26 in the interior of housing 10. N95 filtration material is an electrostatic non-woven polypropylene fiber; a synthetic plastic fiber made out of fossil fuels like oil. This fiber is similar to ones found in clothing like rain jackets, yoga pants, and stretchy fabric. N95 or higher filter material 28 extends to the circumferential wall 16 and over opening 20 of housing 10. In some embodiments, N95 or higher filter material 28 is wafer-shaped. In some embodiments, N95 or higher filter material has a different shape. In some embodiments, N95 or higher filter material 28 extends from the upper surface of filter media 26 and the lowest portion of the domed frontal wall 12.

The effectiveness of the present invention is enhanced by producing nonlinear turbulent air flow within the housing. Turbulence is effectively produced by the pattern of airflow inside the device whereby air enters during inhalation through the multiple side openings 22 and circulates up towards the dead space 24, and then hits the central dimple 29 formed centrally, interiorly of the domed frontal wall 12 and then goes down towards the trachea through the cylindrical conduit between panels 14. The turbulent airflow process is reversed during exhalation. The turbulence assures good humidification and heat transfer of the air as it passes through the filter. Other means to produce turbulence in the air movement within the housing such as vanes within the housing may be used. However, the turbulence must not be excessive so as to produce resistance to air flow. As a matter of fact, we have shown in previous studies that the (turbulent) airflow resistance in this device is actually lower than the (linear) airflow resistance in competitive devices.

The filter also removes particulates from the air. This is very important for a patient having a tracheotomy tube because the normal filtering by the nasal passages is not available.

The present invention is a new low-profile, high-performance heat moisture exchange device 10, based on air recirculation (turbulent air flow rather than linear airflow). This new compact HME takes advantage of its smaller size, and uniquely discrete profile design to maintain a very low visual profile. It sits like a cap over a tracheotomy cannula or a speaking valve. The HME has a diameter of approximately 1 inch and a height of approximately ⅝ inch. In addition, this HME incorporates unique design elements for optimal and efficient air flow and while maintaining a high level of humidification and heat transfer.

Air flows from the trachea (through the tracheotomy tube and/or the speaking valve) and gets redirected from the center of the HME when it encounters the dimpled/curved section in the center of the frontal wall 12. The airflow is directed towards multiple smaller openings that are located on the side walls 16 and/or the bottom walls 14 of the HME housing 10. This recirculation of air promotes flow instability and transition to turbulence, which intensity will increase with the speed of airflow. Turbulent flow, which may naturally occur within the lungs, is chaotic and involves multiple irregular eddies currents (circular currents) of air of many different length scales. When flow is turbulent, particles exhibit additional transverse motion, which results in increased rates of mass, momentum, and heat exchange.

In FIG. 3b, an oxygen port 30 on the side of the base of the HME device allows coupling the HME device with an oxygen catheter, which actively delivers oxygen that flows inside the HME in a turbulent fashion, amplifying the phenomenon of Brownian movement, and further enhancing the phenomena of impaction/Interception/Diffusion. Oxygen port 30 is formed on a portion of circumferential wall 16 and opens a passage for flow between the exterior of housing 10 of the HME device and the interior of the housing. In some embodiments, oxygen port 30 is at a location on circumferential wall 16 to introduce oxygen into the reticulated foam filter 26. In some embodiments, oxygen port 30 is at a location on circumferential wall 16 to introduce oxygen into the reticulated foam filter 26 and then to N95 filter material 28.

One or more embodiments of the present disclosure provide a novel way to protect tracheostomized patients (and people around them) by wearing a heat and moisture exchanger (HME) that has an N95 (or N99 or N100) filter material inside the housing of the device to filter the virus and other germs. In addition to warming and humidifying inspired air, such an HME is expected to reduce airborne transmission of the virus through inhalation and exhalation. In addition, the HME prevents direct finger contact to the cannula of the tracheotomy tube (since the HME covers the tracheostomy tube) hence minimizing the risk of direct surface transmission of the virus. An additional advantage of one or more embodiments is the protection of surrounding people from being infected by exhaled air from a virus-infected tracheostomized patient.

As background information, the filtering mechanism of conventional HMEs that are currently on the market is either a small foam or corrugated paper, with air flowing rapidly through the HME in a linear fashion. Such HMEs were not designed to filter small particles such as viruses, and newly designed HMEs are needed in order to enhance filtration.

One or more embodiments relate to a novel and improved HME which is fit with a unique filtration system designed to enhance protection of the tracheostomized patient against various infectious germs and unwanted viruses (including COVID 19, influenza and other viruses). in addition to hygroscopic media made of the traditional porous reticulated ester-type polyurethane foam, this novel HME includes an additional layer of filtration consisting of a tightly fit N95 (or N99 or N100) wafer. The reticulated foam and the N95 filter (or N99 or N100) are housed in parallel inside an HME frame which is especially designed to redirect air flow in a turbulent fashion. This combination (the N95 filter and reticulated polyurethane foam and the turbulent airflow inside the device) amplifies the filtration effectiveness of this new device as compared to conventional HMEs.

### A Novel HME with N95 (or N99 or N100) Filtration

One or more embodiments are directed to a novel HME which contains a layer of N95 filter material (or N99 or N100) in parallel with reticulated polyurethane foam filter, inside the body of the HME, a device which is especially designed to redirect airflow in a turbulent fashion. The N95 filter wafer is customized in a wafer shape so that to fit precisely and tightly inside the housing of the HME, with a good seal and little air leakage (FIGS. 3a and 3b). This combination (the N95 filter and reticulated polyurethane foam, and the turbulent airflow inside the device) replicate the Brownian movement of air particles that is described in HEPAS filters along with the phenomena of Impaction, Interception, and Diffusion, hence enhancing the filtration effectiveness with regards to small particles such as COVID-19, influenza and other viruses.

N95 filtration material is an electrostatic non-woven polypropylene fiber. In some embodiments, the N95 filtration material is a synthetic plastic fiber made out of fossil fuels like oil. This fiber is similar to ones found in clothing like rain jackets, yoga pants, and stretchy fabric.

The N95 filtration material filters out contaminants like dust, mist and fumes. The minimum size of 0.3 microns of particulates and large droplets won't pass through the barrier, according to the Centers for Disease Control and Prevention (CDC.)

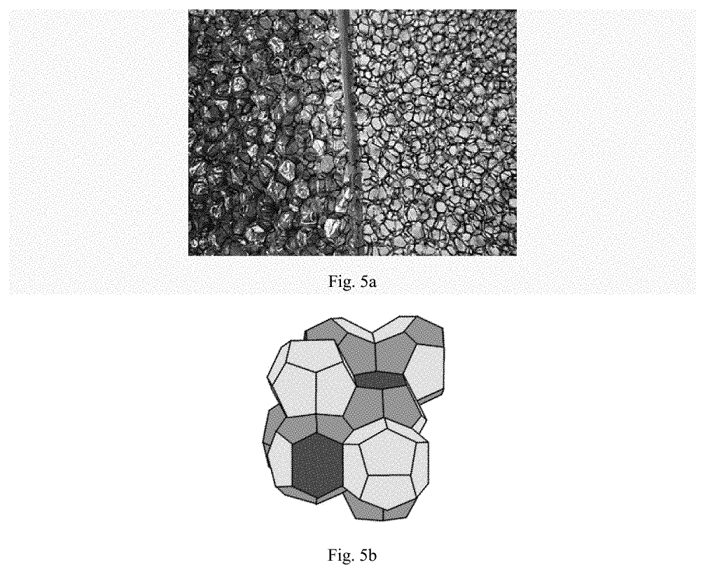

The unique design of the HME redirects airflow inside the device in a turbulent fashion, which substantially increases transverse motion, friction, pressure drag and energy transfer. The dome-shaped outer shell/housing provides additional dead (i.e. empty) space above the foam medium in order to further turbulence and enhance condensation for heat and moisture recapture Importantly, the dimple in the dome center also allows air to recirculate in chaotic and turbulent Eddy currents—circular currents of air of many different length scales, similar to the ones that occur naturally within the lung's alveoli—and air is ultimately redirected out the HME through large openings on the side of the housing. (FIG. 4). The Eddy currents associated with the turbulent airflow inside the HME contain most of the kinetic energy of the turbulent motion. The energy cascades from these large-scale circular structures to smaller and smaller scale structures, eventually creating structures that are small enough that high molecular diffusion and dissipation of energy takes place. The scale at which this happens is known as the Kolmogorov length scale. Air turbulence increases with additional recirculation of air provided by continuous breathing effort, making makes small particles move around inside the filter in a Brownian motion pattern, similar to HEPA, and reproducing the phenomena of Impaction, Interception, and Diffusion that were previously described.

Because turbulent airflow inside the HME replicates Brownian movements (and subsequently the phenomena of impaction/Interception/Diffusion, the HME acts as a mini HEPA filter and can filter particles below the 0.1 micron diameter in size.

As shown in FIG. 3b, the oxygen port on the side of the base of HME allows coupling the HME device with an oxygen catheter, which actively delivers oxygen that flows inside the HME in a turbulent fashion, amplifying the phenomenon of Brownian movement, and further enhancing the phenomena of impaction/Interception/Diffusion.

This method of unpredictable, turbulent HME airflow differs significantly from conventional HMEs on the market, whose design is predicated upon laminar, i.e., linear/streamlined airflow. When airflow is laminar, air generally moves with the same speed and in the same direction. The airflow is smooth and regular, and it follows Bernoulli's Principle, which suggests that a fluid (such as air) traveling over the surface of an object exerts less pressure than if the fluid were still.

The hygroscopic foam inside the HME, is made of reticulated ester-type polyurethane foam impregnated with calcium chloride, which provides an effective filter against unwanted airborne particles. Reticulated polyurethane foam is a versatile, open-cell material that is lightweight, low-odor and highly resistant to mildew. Foam technology involves the manipulation of thousands of plastic bubbles (called cells) of precisely controlled sizes. Reticulation is a post process in foam manufacturing that removes the window membranes of the cell. The cells that make up the foam can have a number of variations, which can also be precisely controlled. Reticulated foam is a very porous, low-density solid foam. The porosity of reticulated foams is vital when designing a custom component or product. ‘Reticulated’ means like a net. Reticulated foams are extremely open foams, i.e., there are few, if any, intact bubbles or cell windows. In contrast, the foam formed by soap bubbles is composed solely of intact (fully enclosed) bubbles. In a reticulated foam, only the lineal boundaries where the bubbles meet (Plateau borders) remain, see FIGS. 5a and 5b.

The combination of turbulent flow, reticulated foam where airflow replicates the Brownian motion previously described, and the addition of an N95 wafer-filter (which is the state-of-the-art filtration mechanism currently available against COVID-19) significantly enhances he filtration potential of the HME, and has the potential offer a much better protection for tracheostomized patients.

One question that begs to be answered is whether placing two filtration mechanisms in parallel inside the HME (N95 wafer in addition to reticulated polyurethane foam) would increase airflow resistance to a point that this would make it difficult for the tracheotomized patients to tolerate the device. We have done in vitro studies to answer this question and found that adding the N95 material does not significantly increase airflow resistance. As a matter of fact, we found that the novel HME, with the N95 wafer and reticulated polyurethane foam, still has a lower airflow resistance as compared to traditional the Mallinckrodt Tracheolife™ II tracheostomy HME, one of the most used HMEs on the market (reference 30).

### Study Comparing Airflow Resistance of Different HMEs

Airflow resistance, as indicated by [hPa] pressure drop, was measured at a flow of 20 liters/minute (or 0.331/sec) which corresponds to the upper limit of light day activity for a tracheotomy patient. The test rig measured air pressure drop (P_{Drop}) and flow (Q) amplitude using a flowmeter attached to a pressurized gas source and a sealed tube that acts as a pneumatic capacitor, which is in turn was connected to the HME device under test. A lumen was placed within the capacitor tube at about mid-level, one side was connected to a Dwyer precision differential manometer, and the other side was open to atmosphere. The flowmeter was adjusted to the target flow rate and the manometer was zeroed with no device connected. The experiment was repeated three time for each device. This method was validated against the method described in ISO 9360.

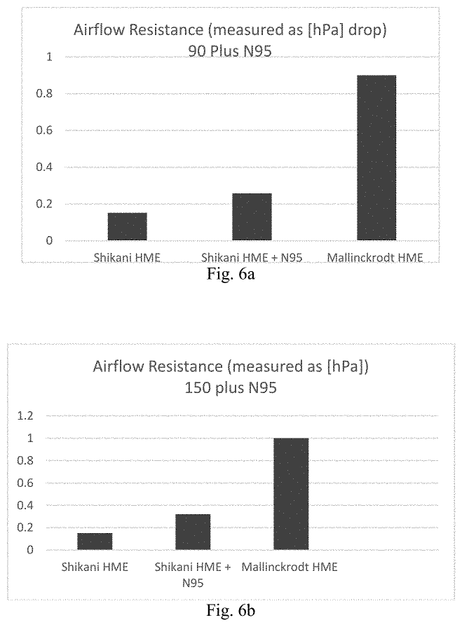

The airflow resistance of the Shikani HME was compared to that of the traditional Mallinckrodt Tracheolife™ II tracheostomy HME, one of the most used HMEs on the market. The tests were done on dry HMEs (before any moisture). Two different thicknesses of N95 wafers were tested inside the Shikani-HME: N95 wafer-90 and N95 wafer-150. The study compared the Shikani-HME (S-HME), the Mallinckrodt Tracheolife™ II tracheostomy HME (M-HME), the S-HME+N95 wafer-90 Plus and the S-HME+N95 wafer-150 Plus. The results showed that the S-HME and the S-HME+N95 (both at 90 Plus and at 150 Plus) had significantly lower resistance as compared to the M-HME (FIGS. 6a and 6b).

Another question that begs to be answered is whether placing the above HME improve tracheal mucosal health.

### Study Comparing Tracheal Mucosal Health of Different HMEs

We have done in vivo studies to answer this question and found that adding the N95 material significantly deceased tracheal mucosal inflammation and infection, and decreases tracheal mucus and crusting, as compared to traditional the Mallinckrodt Tracheolife™ II tracheostomy HME, one of the most used HMEs on the market.

An object of one or more embodiments of the invention is to produce a new HME device with enhanced filtration as compared that traditional HME devices.

A further object of one or more embodiments of the invention is to produce a new HME that includes an N95 filter wafer that is customized in a wafer shape so that to precisely fits inside the housing of the HME, with a good seal and avoiding air leakage.

A further object of one or more embodiments of the invention is to replicate the features of a HEPA filtration inside the small housing of an HME by having the breathed air circulate in a turbulent fashion in a reticulated polyurethane foam, replicating the Brownian movement of HEPA filters.

A further object of one or more embodiments of the invention is to magnify the filtration potential of the HME by adding an N95 filter wafer (or alternatively an N99 or N100) in parallel with reticulated polyurethane foam.

These and other objects will become apparent from a reading of the following specification in conjunction with the enclosed drawings.

One or more embodiments are directed to a novel HME which contains a layer of N95 filter material (or N99 or N100 material) in parallel with reticulated polyurethane foam filter, inside the body of the HME customized in a wafer shape so that to precisely fits inside the housing of the HME, with a good seal and avoiding air leakage. Airflow is redirected inside the HME in a turbulent fashion, replicating the Brownian motion and the phenomena of Impaction, Interception, and Diffusion that are typically found in a HEPA filter, hence enhancing filtration of air that is breathed by tracheotomized patients, and protecting these patients from inhaling airborne germs and viruses, including COVID 19.

In at least one embodiment, the HME device with N95 or higher filter material is a one-time use device which is used for a period of approximately one day by a patient and then replaced with a new HME device. In at least one embodiment, the HME device with N95 or higher filter material is used for a period of greater or lesser time than one day by a patient before being replaced.

In an aspect, a heat and moisture exchanger (HME) device includes: a housing adapted to be received on an end of a tracheotomy tube, the housing having an interior configured to redirect air flow in a turbulent fashion; a reticulated polyurethane foam (RPF) filter material in the housing interior; and an N95 filter material in parallel with the RPF filter material, the N95 filter material having a wafer shape to fit inside the housing of the HME adjacent the RPF filter material, the N95 filter material enhances filtration of air breathed by tracheotomized patients and protects patients from inhaling airborne germs and viruses.

In some embodiments, the N95 filter material is an N99 or N100 filter material.

In some embodiments, the N95 filter material is above the RPF filter material and distal from the end of the housing for receiving the end of the tracheotomy tube.

In some embodiments, the N95 filter material is N95, N99 or N100 material, the N95 filter material being an electrostatic polypropylene material.

In some embodiments, the housing further comprises an oxygen port for receiving a flow of oxygen to the interior of the housing.

In an aspect, a heat and moisture exchanger (HME) device includes: a housing configured to be received on an end of a tracheotomy tube, the housing having: a domed front wall; circumferential walls depending from the domed front wall; a bottom panel joined to the circumferential walls; an opening formed in the bottom panel for receiving the tracheotomy tube therein, the domed front wall having a dimple extending toward the opening in the bottom panel; an inner wall connected to the bottom panel and extending upwardly within the housing toward the domed front wall, the inner wall being substantially parallel to the circumferential walls; and a plurality of spaced-apart openings formed in the circumferential walls; and a filter material stack in the housing interior, the filter material stack including: a first filter material adjacent the bottom panel; and a second filter material adjacent the first filter material, the second filter material different from the first filter material.

In some embodiments, the first filter material covers the entirety of the opening in the bottom panel.

In some embodiments, the first filter material is an N95 or higher filter material.

In some embodiments, the second filter material is a reticulated polyurethane foam filter material.

In some embodiments, the combination of the first filter material and the second filter material are configured to replicate the filtration capability of a HEPA filter in the HME device.

In some embodiments, the circumference of the first filter material is coextensive to the interior of the circumferential walls.

In some embodiments, the first filter material is in a wafer shape.

In some embodiments, a surface of the first filter material distal from the second filter material is in contact with the dimple in the housing interior.

In some embodiments, air flowing through the HME device passes through at least the first filter material.

In some embodiments, air flowing through the HME device passes through the first filter material and the second filter material.

In some embodiments, air flowing into the HME device passes through the second filter material prior to passing through the first filter material.

In some embodiments, the first filter material is an N95 or higher filter material and has a wafer shape in conformity with the interior of the housing of the HME device and thereby providing filtration of air breathed by tracheotomized patients, hence protecting the tracheotomized patients from inhaling airborne germs and viruses, including COVID 19.

In an aspect, a method of using the HME device with a tracheotomy tube wherein the HME device is mounted on the end of the tracheotomy tube includes air being inhaled and exhaled through the tracheotomy tube.

In some embodiments, the air flowing through the HME device passes through at least the N95 filter material.

In some embodiments, the air flowing through the HME device passes through the N95 filter material and the RPF filter material.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A heat and moisture exchanger (HME) device comprising:
a housing adapted to be received on an end of a tracheotomy tube, the housing having an interior configured to redirect air flow in a turbulent fashion;
a reticulated polyurethane foam (RPF) filter material in the housing interior;
an N95 filter material in parallel with the RPF filter material, the N95 filter material having a wafer shape to fit inside the housing of the HME adjacent the RPF filter material, the N95 filter material enhances filtration of air breathed by tracheotomized patients and protects patients from inhaling airborne germs and viruses.

2. The HME device of claim 1, wherein the N95 filter material is an N99 or N100 filter material, the N95 filter material being an electrostatic polypropylene material.

3. The HME device of claim 1, wherein the N95 filter material is above the RPF filter material and distal from the end of the housing for receiving the end of the tracheotomy tube.

4. The HME device of claim 1, wherein the housing further comprises an oxygen port for receiving a flow of oxygen to the interior of the housing.

5. A heat and moisture exchanger (HME) device comprising:
a housing configured to be received on an end of a tracheotomy tube, the housing having:
a domed front wall;
circumferential walls depending from the domed front wall;
a bottom panel joined to the circumferential walls;
an opening formed in the bottom panel for receiving the tracheotomy tube therein, the domed front wall having a dimple extending toward the opening in the bottom panel;
an inner wall connected to the bottom panel and extending upwardly within the housing toward the domed front wall, the inner wall being substantially parallel to the circumferential walls; and
a plurality of spaced-apart openings formed in the circumferential walls; and
a filter material stack in the housing interior, the filter material stack comprising:
a first filter material adjacent the bottom panel; and
a second filter material adjacent the first filter material, the second filter material different from the first filter material.

6. The HME device of claim 5, wherein the first filter material covers the entirety of the opening in the bottom panel.

7. The HME device of claim 5, wherein the first filter material is an N95 or higher filter material.

8. The HME device of claim 7, wherein the second filter material is a reticulated polyurethane foam filter material.

9. The HME device of claim 5, wherein the combination of the first filter material and the second filter material are configured to replicate the filtration capability of a HEPA filter in the HME device, wherein turbulent airflow inside the HME replicates Brownian movements (and subsequently the phenomena of Impaction/Interception/Diffusion that are characteristic of a HEPA filter).

10. The HME device of claim 5 wherein an oxygen port is added to the housing and allows coupling the HME device with an oxygen catheter which actively delivers oxygen that flows inside the HME device in a turbulent fashion, (hence amplifying the phenomenon of Brownian movement, and further enhancing the phenomena of Impaction/Interception/Diffusion that are characteristic of a HEPA filter).

11. The HME device of claim 6, wherein the circumference of the first filter material is coextensive to the interior of the circumferential walls.

12. The HME device of claim 5, wherein the first filter material is in a wafer shape.

13. The HME device of claim 12, wherein a surface of the first filter material distal from the second filter material is in contact with the dimple in the housing interior.

14. The HME device of claim 5, wherein air flowing through the HME device passes through at least the first filter material.

15. The HME device of claim 14, wherein air flowing through the HME device passes through the first filter material and the second filter material.

16. The HME device of claim 15, wherein air flowing into the HME device passes through the second filter material prior to passing through the first filter material.

17. The HME device of claim 5, wherein the first filter material is an N95 or higher filter material and has a wafer shape in conformity with the interior of the housing of the HME device and thereby providing filtration of air breathed by tracheotomized patients, hence protecting the tracheotomized patients from inhaling airborne germs and viruses, including COVID 19.

18. A method of using the HME device of claim 1 with a tracheotomy tube wherein the HME device is mounted on the end of the tracheotomy tube and filters air being inhaled and exhaled through the tracheotomy tube.

19. The method of claim 18, wherein the air flowing through the HME device passes through at least the N95 filter material.

20. The method of claim 18, wherein the air flowing through the HME device passes through the N95 filter material and the RPF filter material.
